SEQUENCE LISTING (19 sequences)

SEQ ID NO: 1 - DNA, 2166 bp, Escherichia coli
atgaacgttattgcaatattgaatcacatgggggtttattttaaagaagaacccatccgtgaacttcatcgcgcgcttgaacgtctgaacttccagattgtttacccgaacgaccgtgacgacttattaaaactgatcgaaaacaatgcgcgtctgtgcggcgttatttttgactgggataaatataatctcgagctgtgcgaagaaattagcaaaatgaacgagaacctgccgttgtacgcgttcgctaatacgtattccactctcgatgtaagcctgaatgacctgcgtttacagattagcttctttgaatatgcgctgggtgctgctgaagatattgctaataagatcaagcagaccactgacgaatatatcaacactattctgcctccgctgactaaagcactgtttaaatatgttcgtgaaggtaaatatactttctgtactcctggtcacatgggcggtactgcattccagaaaagcccggtaggtagcctgttctatgatttctttggtccgaataccatgaaatctgatatttccatttcagtatctgaactgggttctctgctggatcacagtggtccacacaaagaagcagaacagtatatcgctcgcgtctttaacgcagaccgcagctacatggtgaccaacggtacttccactgcgaacaaaattgttggtatgtactctgctccagcaggcggcaccattctgattgaccgtaactgccacaaatcgctgacccacctgatgatgatgagcgatgttacgccaatctatttccgcccgacccgtaacgcttacggtattcttggtggtatcccacagagtgaattccagcacgctaccattgctaagcgcgtgaaagaaacaccaaacgcaacctggccggtacatgctgtaattaccaactctacctatgatggtctgctgtacaacaccgacttcatcaagaaaacactggatgtgaaatccatccactttgactccgcgtgggtgccttacaccaacttctcaccgatttacgaaggtaaatgcggtatgagcggtggccgtgtagaagggaaagtgatttacgaaacccagtccactcacaaactgctggcggcgttctctcaggcttccatgatccacgttaaaggtgacgtaaacgaagaaacctttaacgaagcctacatgatgcacaccaccacttctccgcactacggtatcgtggcgtccactgaaaccgctgcggcgatgatgaaaggcaatgcaggtaagcgtctgattaacggttctattgaacgtgcgatcaaattccgtaaagagatcaaacgtctgagaacggaatctgatggctggttctttgatgtatggcagccggatcatatcgatacgactgaatgctggccgctgcgtcctgacagcacctggcacggcttcaaaaacatcgataacgagcacatgtatcttgacccgatcaaagtcaccctgctgactccggggatggaaaaagacggcaccatgagcgactttggtattccggccagcatcgtggcgaaatacctcgacgaacatggcatcgttgttgagaaaaccggtccgtataacctgctgttcctgttcagcatcggtatcgataagaccaaagcactgagcctgctgcgtgctctgactgactttaaacgtgcgttcgacctgaacctgcgtgtgaaaaacatgctgccgtctctgtatcgtgaagatcctgaattctatgaaaacatgcgtattcaggaactggctcagaatatccacaaactgattgttcaccacaatctgccggatctgatgtatcgcgcatttgaagtgctgccgacgatggtaatgactccgtatgctgcattccagaaagagctgcacggtatgaccggagaagtttacctcgacgaaatggtaggtcgtattaacgccaatatgatccttccgtacccgccgggagttcctctggtaatgccgggtgaaatgatcaccgaagaaagccgtccggttctggagttcctgcagatgctgtgtgaaatcggcgctcactatccgggctttgaaaccgatattcacggtgcataccgtcaggctgatggccgctataccgttaaggtattgaaagaagaaagcaaaaaaaagcttcatgcggccgcataa

SEQ ID NO: 2 - DNA, 657 bp, Escherichia coli
atgtctgataacgacgaattgcagcaaatcgcgcatctgcgccgtgaatacaccaaaggcgggttacgccgccgcgatcttcccgccgatccattaaccctttttgaacgttggctctctcaggcttgtgaagccaaactggcggaccctaccgcgatggtggtcgctaccgtggatgaacatggtcagccttatcagcgcatcgttttactcaaacattacgacgaaaaaggcatggtgttttacaccaacctcggcagccgtaaagcacatcaaatcgaaaataatccgcgcgttagcctgctgttcccgtggcatacccttgagcgccaggtgatggtgatcggtaaagcagaacgactttcgactctcgaagtgatgaaatattttcatagccgcccgcgtgatagccagattggtgcatgggtttcgaagcagtccagtcgcatttctgcccgcggtatccttgaaagtaaattcctggagctgaagcagaagtttcaacagggcgaagtgccattgccgagcttttggggcggttttcgcgtcagccttgaacagattgagttctggcagggtggtgagcatcgcctgcatgaccgctttttgtaccagcgtgaaaatgatgcgtggaagattgatcgtcttgcaccctga

SEQ ID NO: 3 - DNA, 852 bp, Escherichia coli
ttatgcttccgccagcggcggcaaaatcaattcatcgctctcatgctgctgggtgtagcgcatcacttccagtacgcgcaaccccgctcggtgcactgcatcggttaacgccttccctttcagcaagccactgatgagctgagcacaaaacaggtcgccagtccctttcaggtcggtttttacccgtgaatgggaaatgacattcacgctgtcggcagtgaccaccacaacctgcatctcctgattttcttcattaccggaggcgctggtaaccaccacccattttaatgtgtctgaaagcagactttttgcggcagcaatggcactgtcgagatcgcggcaatttttaccggtcaggatttccaactcaaagatattgggggtaattccctgcgccagcggcagtaaatattgtcgatacgcttcgggaaggtcaggtttgacataaattccgctatcaatatcgccaatcaccggatcgaccatgatcaataggtcaggatggtctttgcgtagcgcagtcagccactcggcaaggattttgatttgcgatgccgttcccatatagcccgtggttacagcacgaagttggcgcagcgcatcacgctcctgaagcgcacgcaaatagccgctaaaccattcgtccggaatcgcaccaccgtagaaagtgtcataatgcggcgtattgctcagcaataccgtcggcacggcaaagacattcaggccgttctgtttgatagcaggcacggcaatgctgttgcccacgctgccgtaaaccacctgcgactgcacggcgacgatatccgcctgcagtgccctactcttatcgttaaacaacaacaaactactcat

SEQ ID NO: 4 - DNA, 864 bp, Escherichia coli
atgatgaaaaatattctcgctatccagtctcacgttgtttatggtcatgcgggtaacagtgcggcagagtttccgatgcgccgcctgggcgcgaacgtctggccgctgaacaccgttcaattttctaatcacacccaatacggcaaatggactggctgcgtgatgccgcccagccatttaaccgaaattgtgcaaggcattgccgccattgataaattacacacctgtgatgccgtattaagtggctatctgggatcggcggagcagggtgaacatatcctcggtatcgtccgtcaggtgaaagccgcgaatccgcaggcgaaatatttttgcgatccggtaatgggtcatccggaaaaaggctgtatcgttgcaccgggtgtcgcagagtttcatgtgcggcacggtttgcctgccagcgatatcattgcgccaaatctggttgagctggaaatactctgtgagcatgcggtaaataacgtcgaagaagcggttctggcagcgcgcgaactcattgcgcaagggccacaaattgtgttggttaaacacctggcgcgagctggctacagccgtgaccgttttgaaatgctgctggtcaccgccgatgaagcctggcatatcagccgtccgctggtggattttggtatgcgccagccggtaggtgttggtgatgtgacgagcggtttactgctggtgaaaccgcttcagggggcaacgctgcaggaggcgctggaacatgtgaccgctgcagtctacgaaatcatggtgaccaccaaagcaatgcaggaatatgagctgcaagtggtggctgctcaggatcgtattgccaaaccagaacattacttcagcgcaacaaagctctga

SEQ ID NO: 5 - DNA, 35 bp, Artificial Sequence
  J23100 promoter
ttgacggctagctcagtcctaggtacagtgctagc

SEQ ID NO: 6 - DNA, 200 bp, Artificial Sequence
  PlacI promoter
caatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacaca

SEQ ID NO: 7 - DNA, 5926 bp, Artificial Sequence
  pSIT-PT7-CadA
tcccttatgcgactcctgcattaggaaattaatacgactcactataggggaattgtgagcggataacaattcccctgtagaaataattttgtttaactttaataaggagatataccatgtctgaattcgacggatccatgaacgttattgcaatattgaatcacatgggggtttattttaaagaagaacccatccgtgaacttcatcgcgcgcttgaacgtctgaacttccagattgtttacccgaacgaccgtgacgacttattaaaactgatcgaaaacaatgcgcgtctgtgcggcgttatttttgactgggataaatataatctcgagctgtgcgaagaaattagcaaaatgaacgagaacctgccgttgtacgcgttcgctaatacgtattccactctcgatgtaagcctgaatgacctgcgtttacagattagcttctttgaatatgcgctgggtgctgctgaagatattgctaataagatcaagcagaccactgacgaatatatcaacactattctgcctccgctgactaaagcactgtttaaatatgttcgtgaaggtaaatatactttctgtactcctggtcacatgggcggtactgcattccagaaaagcccggtaggtagcctgttctatgatttctttggtccgaataccatgaaatctgatatttccatttcagtatctgaactgggttctctgctggatcacagtggtccacacaaagaagcagaacagtatatcgctcgcgtctttaacgcagaccgcagctacatggtgaccaacggtacttccactgcgaacaaaattgttggtatgtactctgctccagcaggcggcaccattctgattgaccgtaactgccacaaatcgctgacccacctgatgatgatgagcgatgttacgccaatctatttccgcccgacccgtaacgcttacggtattcttggtggtatcccacagagtgaattccagcacgctaccattgctaagcgcgtgaaagaaacaccaaacgcaacctggccggtacatgctgtaattaccaactctacctatgatggtctgctgtacaacaccgacttcatcaagaaaacactggatgtgaaatccatccactttgactccgcgtgggtgccttacaccaacttctcaccgatttacgaaggtaaatgcggtatgagcggtggccgtgtagaagggaaagtgatttacgaaacccagtccactcacaaactgctggcggcgttctctcaggcttccatgatccacgttaaaggtgacgtaaacgaagaaacctttaacgaagcctacatgatgcacaccaccacttctccgcactacggtatcgtggcgtccactgaaaccgctgcggcgatgatgaaaggcaatgcaggtaagcgtctgattaacggttctattgaacgtgcgatcaaattccgtaaagagatcaaacgtctgagaacggaatctgatggctggttctttgatgtatggcagccggatcatatcgatacgactgaatgctggccgctgcgtcctgacagcacctggcacggcttcaaaaacatcgataacgagcacatgtatcttgacccgatcaaagtcaccctgctgactccggggatggaaaaagacggcaccatgagcgactttggtattccggccagcatcgtggcgaaatacctcgacgaacatggcatcgttgttgagaaaaccggtccgtataacctgctgttcctgttcagcatcggtatcgataagaccaaagcactgagcctgctgcgtgctctgactgactttaaacgtgcgttcgacctgaacctgcgtgtgaaaaacatgctgccgtctctgtatcgtgaagatcctgaattctatgaaaacatgcgtattcaggaactggctcagaatatccacaaactgattgttcaccacaatctgccggatctgatgtatcgcgcatttgaagtgctgccgacgatggtaatgactccgtatgctgcattccagaaagagctgcacggtatgaccggagaagtttacctcgacgaaatggtaggtcgtattaacgccaatatgatccttccgtacccgccgggagttcctctggtaatgccgggtgaaatgatcaccgaagaaagccgtccggttctggagttcctgcagatgctgtgtgaaatcggcgctcactatccgggctttgaaaccgatattcacggtgcataccgtcaggctgatggccgctataccgttaaggtattgaaagaagaaagcaaaaaaaagcttcatgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctc

SEQ ID NO: 8 - DNA, 2154 bp, Artificial Sequence
  pSU
actagtagcggccgctgcagtccggcaaaaaagggcaaggtgtcaccaccctgccctttttctttaaaaccgaaaagattacttcgcgttatgcagatctattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagtcgacaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacctgacgtctaagaaaccattattatcatgacattaacctataaaaataggcgtatcacgaggcagaatttcagataaaaaaaatccttagctttcgctaaggatgatttctggaattcgcggccgcttctaga

SEQ ID NO: 9 - DNA, 3230 bp, Artificial Sequence
  pPK
caatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggatccaaagaggagaaaaagcttatgagtagtttgttgttgtttaacgataagagtagggcactgcaggcggatatcgtcgccgtgcagtcgcaggtggtttacggcagcgtgggcaacagcattgccgtgcctgctatcaaacagaacggcctgaatgtctttgccgtgccgacggtattgctgagcaatacgccgcattatgacactttctacggtggtgcgattccggacgaatggtttagcggctatttgcgtgcgcttcaggagcgtgatgcgctgcgccaacttcgtgctgtaaccacgggctatatgggaacggcatcgcaaatcaaaatccttgccgagtggctgactgcgctacgcaaagaccatcctgacctattgatcatggtcgatccggtgattggcgatattgatagcggaatttatgtcaaacctgaccttcccgaagcgtatcgacaatatttactgccgctggcgcagggaattacccccaatatctttgagttggaaatcctgaccggtaaaaattgccgcgatctcgacagtgccattgctgccgcaaaaagtctgctttcagacacattaaaatgggtggtggttaccagcgcctccggtaatgaagaaaatcaggagatgcaggttgtggtggtcactgccgacagcgtgaatgtcatttcccattcacgggtaaaaaccgacctgaaagggactggcgacctgttttgtgctcagctcatcagtggcttgctgaaagggaaggcgttaaccgatgcagtgcaccgagcggggttgcgcgtactggaagtgatgcgctacacccagcagcatgagagcgatgaattgattttgccgccgctggcggaagcataaactagtagcggccgctgcagtccggcaaaaaagggcaaggtgtcaccaccctgccctttttctttaaaaccgaaaagattacttcgcgttatgcagatctattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagtcgacaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacctgacgtctaagaaaccattattatcatgacattaacctataaaaataggcgtatcacgaggcagaatttcagataaaaaaaatccttagctttcgctaaggatgatttctggaattcgcggccgcttctaga

SEQ ID NO: 10 - DNA, 3077 bp, Artificial Sequence
  pJY
ttgacggctagctcagtcctaggtacagtgctagcggatccaaagaggagaaaaagcttatgatgaaaaatattctcgctatccagtctcacgttgtttatggtcatgcgggtaacagtgcggcagagtttccgatgcgccgcctgggcgcgaacgtctggccgctgaacaccgttcaattttctaatcacacccaatacggcaaatggactggctgcgtgatgccgcccagccatttaaccgaaattgtgcaaggcattgccgccattgataaattacacacctgtgatgccgtattaagtggctatctgggatcggcggagcagggtgaacatatcctcggtatcgtccgtcaggtgaaagccgcgaatccgcaggcgaaatatttttgcgatccggtaatgggtcatccggaaaaaggctgtatcgttgcaccgggtgtcgcagagtttcatgtgcggcacggtttgcctgccagcgatatcattgcgccaaatctggttgagctggaaatactctgtgagcatgcggtaaataacgtcgaagaagcggttctggcagcgcgcgaactcattgcgcaagggccacaaattgtgttggttaaacacctggcgcgagctggctacagccgtgaccgttttgaaatgctgctggtcaccgccgatgaagcctggcatatcagccgtccgctggtggattttggtatgcgccagccggtaggtgttggtgatgtgacgagcggtttactgctggtgaaactgcttcagggggcaacgctgcaggaggcgctggaacatgtgaccgctgcagtctacgaaatcatggtgaccaccaaagcaatgcaggaatatgagctgcaagtggtggctgctcaggatcgtattgccaaaccagaacattacttcagcgcaacaaagctctgaactagtagcggccgctgcagtccggcaaaaaagggcaaggtgtcaccaccctgccctttttctttaaaaccgaaaagattacttcgcgttatgcagatctattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagtcgacaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacctgacgtctaagaaaccattattatcatgacattaacctataaaaataggcgtatcacgaggcagaatttcagataaaaaaaatccttagctttcgctaaggatgatttctggaattcgcggccgcttctaga

SEQ ID NO: 11 - DNA, 30 bp, Artificial Sequence
  primer JH/PH-F
gcaagcttatgtctgataacgacgaattgc

SEQ ID NO: 12 - DNA, 30 bp, Artificial Sequence
  primer JH/PH-R
ctactagttcagggtgcaagacgatcaatc

SEQ ID NO: 13 - DNA, 31 bp, Artificial Sequence
  primer JY/PY-F
gcgcttatgatgaaaaatattctcgctatcc

SEQ ID NO: 14 - DNA, 31 bp, Artificial Sequence
  primer JY/PY-R
ctactagttcagagctttgttgcgctgaagt

SEQ ID NO: 15 - DNA, 31 bp, Artificial Sequence
  primer JK/PK-F
gcgcttatgagtagtttgttgttgtttaacg

SEQ ID NO: 16 - DNA, 30 bp, Artificial Sequence
  primer JK/PK-R
actagtttatgcttccgccagcggcggcaa

SEQ ID NO: 17 - DNA, 32 bp, Artificial Sequence
  primer pSIT-PT7-CadA-F
gcggatccatgaacgttattgcaatattgaat

SEQ ID NO: 18 - DNA, 32 bp, Artificial Sequence
  primer pSIT-PT7-CadA-R
gcaagcttttttttgctttcttctttcaatac

SEQ ID NO: 19 - DNA, 4267 bp, Artificial Sequence
  pSU-J23100-CadA
ttgacggctagctcagtcctaggtacagtgctagcggatccaaagaggagaaaaagcttatgaacgttattgcaatattgaatcacatgggggtttattttaaagaagaacccatccgtgaacttcatcgcgcgcttgaacgtctgaacttccagattgtttacccgaacgaccgtgacgacttattaaaactgatcgaaaacaatgcgcgtctgtgcggcgttatttttgactgggataaatataatctcgagctgtgcgaagaaattagcaaaatgaacgagaacctgccgttgtacgcgttcgctaatacgtattccactctcgatgtaagcctgaatgacctgcgtttacagattagcttctttgaatatgcgctgggtgctgctgaagatattgctaataagatcaagcagaccactgacgaatatatcaacactattctgcctccgctgactaaagcactgtttaaatatgttcgtgaaggtaaatatactttctgtactcctggtcacatgggcggtactgcattccagaaaagcccggtaggtagcctgttctatgatttctttggtccgaataccatgaaatctgatatttccatttcagtatctgaactgggttctctgctggatcacagtggtccacacaaagaagcagaacagtatatcgctcgcgtctttaacgcagaccgcagctacatggtgaccaacggtacttccactgcgaacaaaattgttggtatgtactctgctccagcaggcggcaccattctgattgaccgtaactgccacaaatcgctgacccacctgatgatgatgagcgatgttacgccaatctatttccgcccgacccgtaacgcttacggtattcttggtggtatcccacagagtgaattccagcacgctaccattgctaagcgcgtgaaagaaacaccaaacgcaacctggccggtacatgctgtaattaccaactctacctatgatggtctgctgtacaacaccgacttcatcaagaaaacactggatgtgaaatccatccactttgactccgcgtgggtgccttacaccaacttctcaccgatttacgaaggtaaatgcggtatgagcggtggccgtgtagaagggaaagtgatttacgaaacccagtccactcacaaactgctggcggcgttctctcaggcttccatgatccacgttaaaggtgacgtaaacgaagaaacctttaacgaagcctacatgatgcacaccaccacttctccgcactacggtatcgtggcgtccactgaaaccgctgcggcgatgatgaaaggcaatgcaggtaagcgtctgattaacggttctattgaacgtgcgatcaaattccgtaaagagatcaaacgtctgagaacggaatctgatggctggttctttgatgtatggcagccggatcatatcgatacgactgaatgctggccgctgcgtcctgacagcacctggcacggcttcaaaaacatcgataacgagcacatgtatcttgacccgatcaaagtcaccctgctgactccggggatggaaaaagacggcaccatgagcgactttggtattccggccagcatcgtggcgaaatacctcgacgaacatggcatcgttgttgagaaaaccggtccgtataacctgctgttcctgttcagcatcggtatcgataagaccaaagcactgagcctgctgcgtgctctgactgactttaaacgtgcgttcgacctgaacctgcgtgtgaaaaacatgctgccgtctctgtatcgtgaagatcctgaattctatgaaaacatgcgtattcaggaactggctcagaatatccacaaactgattgttcaccacaatctgccggatctgatgtatcgcgcatttgaagtgctgccgacgatggtaatgactccgtatgctgcattccagaaagagctgcacggtatgaccggagaagtttacctcgacgaaatggtaggtcgtattaacgccaatatgatccttccgtacccgccgggagttcctctggtaatgccgggtgaaatgatcaccgaagaaagccgtccggttctggagttcctgcagatgctgtgtgaaatcggcgctcactatccgggctttgaaaccgatattcacggtgcataccgtcaggctgatggccgctataccgttaaggtattgaaagaagaaagcaaaaaatgaagatctattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagtcgacaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgagtgccacctgacgtctaagaaaccattattatcatgacattaacctataaaaataggcgtatcacgaggcagaatttcagataaaaaaaatccttagctttcgctaaggatgatttctggaattcgcggccgcttctaga

(19) United States
(12) Patent Application Publication
(10) Pub. No.: US 2022/0380745 A1
(43) Pub. Date: Dec. 1, 2022

(54) RECOMBINANT MUTANT MICROORGANISM AND METHOD FOR PRODUCING CADAVERINE BY USING SAME MICROORGANISM

(71) Applicant: CHINA PETROCHEMICAL DEVELOPMENT CORPORATION, TAIPEI (TAIWAN), Kaohsiung City (TW)

(72) Inventors: Jo-Shu CHANG, Kaohsiung City (TW); I-Son NG, Kaohsiung City (TW); Cheng-Feng XUE, Kaohsiung City (TW); Ying-Jung CHEN, Kaohsiung City (TW); Chang-Yi CHEN, Kaohsiung City (TW); Yu-Chiao LIU, Kaohsiung City (TW)

(73) Assignee: CHINA PETROCHEMICAL DEVELOPMENT CORPORATION, TAIPEI (TAIWAN), Kaohsiung City (TW)

(21) Appl. No.: 17/333,141

(22) Filed: May 28, 2021

(51) Int. Cl. C12N 9/88; C12N 9/12; C12P 13/00

(52) U.S. Cl. CPC C12N 9/88; C12Y 401/01018; C12Y 207/01035; C12N 9/1205; C12P 13/001

(57) ABSTRACT

Provided is a non-naturally occurring microorganism capable of producing cadaverine, wherein the microorganism is genetically modified to overexpress lysine decarboxylase and pyridoxal kinase. Also provided is a method for producing cadaverine by using such microorganism without adding external pyridoxal 5′-phosphate.

### BACKGROUND

### 1. Technical Field

The present disclosure relates to a cell biocatalyst, and more particularly, to a recombinant microorganism used as a whole cell biocatalyst, and a method for producing cadaverine by using the recombinant microorganism.

### 2. Description of Associated Art

Recently, bio-polymers are with great interests not only in the industry but also for academic research because of the desire in reducing carbon dioxide from petrochemical processes, as well as increasing importance of alternative and sustainable production of polymeric and composite materials [1, 2, 3]. Cadaverine (also known as 1,5-diaminopentane) is used as an essential biochemical compound, particularly in the reaction with dicarboxylic acid to obtain polyamides [4]. Among all, polyamide 54 (PA54) is formed by the polycondensation of cadaverine and succinic acid that is expected as a critical bio-based chemical production to replace conventional petroleum-based polyamides with an annual global market of 3.5 million tons [5].

Cadaverine can be enzymatically synthesized from lysine via lysine decarboxylase (EC 4.1.1.18) by whole-cell bioconversion or direct microbial fermentation utilizing lysine-producing microorganisms [6, 7]. However, such enzyme requires the cofactor, pyridoxal-5′-phosphate (PLP), and the production of cadaverine also highly relies on the amount of PLP [8, 9]. Therefore, Ma and his colleagues engineered a lysine decarboxylase CadA-overexpressing E. coli strain by introducing the ribose 5-phosphate (R5P)-dependent pathway genes pdxS and pdxT from Bacillus subtilis [10]. As a result, the intracellular PLP concentration reached up to 1144 nmol/g dry cell weight (DCW), along with an increase in specific cadaverine production of 25 g/g-DCW/h. On the other hand, Kim et al. reported the highest conversion of pyridoxal (PL) to PLP by pyridoxal kinase (PdxY) [11], resulting in 80% yield of 0.4 M lysine to cadaverine in whole-cell biotransformation. Also, Moon et al. introduced an ATP regeneration system by using polyphosphate kinase (PPK) into CadA and PdxY system for a sufficient supply of PLP, with the addition of hexadecyl-trimethyl-ammonium bromide to enhance cadaverine production [9].

However, there is still an unmet need for a high-conversion rate and high-yielding method for PLP-assisted cadaverine production by an in vivo to in vitro biotransformation.

### SUMMARY

In view of the foregoing, the present disclosure provides a recombinant microorganism for producing cadaverine, which is genetically modified and thus capable of overexpressing lysine decarboxylase and pyridoxal kinase.

In at least one embodiment of the present disclosure, the recombinant microorganism comprises a first nucleotide sequence encoding a first protein having lysine decarboxylase activity and a second nucleotide sequence encoding a second protein having pyridoxal kinase activity. In some embodiments, the first nucleotide sequence comprises a sequence having at least 80% identity to SEQ ID NO. 1, and the second nucleotide sequence comprises a sequence having at least 80% identity to one of SEQ ID NOs. 2 to 4.

In at least one embodiment of the present disclosure, the first protein has the activity of lysine decarboxylase 1 (CadA), and the second protein has the activity of pyridoxal kinase selected from the group consisting of PdxH, PdxK, PdxY, and a combination thereof.

In at least one embodiment of the present disclosure, the second nucleotide sequence further comprises a constitutive promoter for regulating expression of the second protein. In some embodiments, the constitutive promoter is selected from the group consisting of J23100, J23101, J23102, J23103, J23104, J23105, J23106, J23107, J23108, J23109, J23110, J23111, J23112, J23113, J23114, J23115, J23116, J23117, J23118, J23119, and PlacI. In some embodiments, the constitutive promoter is J23100 or PlacI.

In at least one embodiment of the present disclosure, at least one of the first nucleotide sequence and the second nucleotide sequence is carried in an expression vector of the recombinant microorganism. In some embodiments of the present disclosure, the recombinant microorganism has at least one expression vector comprising the first nucleotide sequence.

In at least one embodiment of the present disclosure, the first nucleotide sequence and the second nucleotide sequence are carried in different expression vectors of the recombinant microorganism. In some embodiments of the present disclosure, the first nucleotide sequence and the second nucleotide sequence are carried in the same expression vector of the recombinant microorganism.

In at least one embodiment of the present disclosure, the recombinant microorganism is a recombinant microorganism of Escherichia, Klebsiella, Erwinia, Serratia, Providencia, Corynebacterium, or Brevibacterium. In some embodiments, the recombinant microorganism is a recombinant Escherichia coli (E. coli) strain. In some embodiments, the recombinant microorganism may be, but not limited to, recombinant E. coli BL21 or E. coli BL21(DE3).

In at least one embodiment of the present disclosure, the recombinant microorganism has been cold-shocked. In some embodiments, the recombinant microorganism has been exposed to a cold temperature, such as −80° C. to 25° C.

In at least one embodiment of the present disclosure, the recombinant microorganism is a recombinant E. coli strain JcadA-Wmut, deposited in DSME-DEUTSCHE SAMMLUNG VON MIKROORGANISMEN UND ZELLKULTUREN GmbH (DSMZ) under Accession No. DSM 33754.

The present disclosure also provides a method for producing cadaverine, comprising culturing a microorganism for producing cadaverine in a medium without lysine, mixing the cultured microorganism with lysine in a solution to perform bioconversion of the lysine to the cadaverine, and recovering the cadaverine from the solution, wherein the microorganism is capable of overexpressing a protein having lysine decarboxylase activity and a protein having pyridoxal kinase activity.

In at least one embodiment of the present disclosure, the medium comprises an inducer capable of inducing expression of the protein having lysine decarboxylase activity in the microorganism. In some embodiments, the inducer is isopropyl β-d-1-thiogalactopyranoside (IPTG). In some embodiments, the inducer is in a concentration of 5 μM to 50 μM.

In at least one embodiment of the present disclosure, the method further comprises cold shocking the cultured microorganism before mixing with lysine, wherein the cold shocking comprises storing the cultured microorganism at −80° C. to 25° C. for 1 hour to 10 days. In some embodiments, the cold shocking comprises storing the cultured microorganism at −20° C. for 2 days.

In at least one embodiment of the present disclosure, the medium further comprises a substrate of the pyridoxal kinase. In at least one embodiment of the present disclosure, the substrate is selected from the group consisting of pyridoxamine (PM), pyridoxine (PN), pyridoxal (PL), pyridoxamine-5′-phosphate (PMP), pyridoxine-5′-phosphate (PNP), and any combination thereof. In some embodiments, the substrate is in a concentration of from 0.1 μM to 0.5 mM.

In at least one embodiment of the present disclosure, the method further comprises separating the cultured microorganism from the medium to form a whole-cell biocatalyst for producing cadaverine. In some embodiments, the method further comprises adding the separated cultured microorganism into the solution to perform the mixing.

In at least one embodiment of the present disclosure, for the step of mixing, the concentration of lysine in the solution is from 0.1 M to 2.0 M, and the concentration of the microorganism is represented by OD₆₀₀ of at least 0.5.

In the present disclosure, it is provided with a recombinant microorganism having increased ability to intracellularly produce PLP as compared to an unmodified wild-type microorganism. Therefore, the recombinant microorganism provided in the present disclosure may be used as a whole-cell biocatalyst with the improved effectivity of producing cadaverine without adding external pyridoxal 5′-phosphate, and thus has an immense potential in future industrial applications.

### BRIEF DESCRIPTION OF THE DRAWINGS

The present disclosure can be more fully understood by reading the following descriptions of the embodiments, with reference made to the accompanying drawings.

FIGS. 1A and 1B show the construction maps of plasmids pSIT-P_{T7}-CadA and pSU-P-GOI. P_{T7}: T7 promotor; Km^{R}: kanamycin resistance gene; HindIII, BamHI, EcoRI, and SpeI: recognition sites of restriction enzymes; RSF Ori: RSF origin of replication; pUC Ori: pUC origin of replication; CadA: lysine decarboxylase CadA gene; GOI: gene of interest; pdxH, pdxY, and pdxK: pyridoxal kinase genes; Cm^{R}: chloramphenicol acetyltransferase gene; RBS: ribosome-binding site.

FIG. 2 shows cell growth curves of wild type strains W3110 and BL21, or W3110 and BL21 strains harboring the genes pdxH, pdxK, and pdxY, which are driven by J23100 or PlacI promoter in Luria-Bertani (LB) medium with 0.2 mM precursors of PL (upper) and PN (lower).

FIG. 3A shows the result of sodium dodecyl sulfate polyacrylamide gel electrophoresis (SDS-PAGE) analysis of strains DH5a, W3110, and BL21 harboring the genes pdxH, and pdxY driven by the J23100 or PlacI promoter in Luria-Bertani (LB) medium from the precursor PN (upper) and PL (lower). Lane 1: wild type (WT); Lane 2: protein marker (M); Lane 3: pdxH driven by the J23100 promoter (JH); Lane 4: pdxH driven by the PlacI promoter (PH); Lane 5: pdxY driven by the J23100 promoter (JY); Lane 6: pdxY driven by the PlacI promoter (PY); Lane 7: pdxK driven by the J23100 promoter (JK); Lane 8: pdxK driven by the PlacI promoter (PK).

FIG. 3B shows the intracellular PLP concentrations of the strain BL21 harboring different plasmids (JH, PH, JY, PY, JK, and PK) with 0.2 mM of PN or PL as the precursor.

FIGS. 4A to 4F show the effect of the pdxY or pdxK gene on cell growth, cadaverine production, and intracellular PLP levels in strains A, AJY, and APK. FIG. 4A shows cell growth with 0.2 mM PN and 50 g/L lysine in LB; FIG. 4B shows cadaverine production by 50 g/L lysine; FIG. 4C shows PLP concentration in LB with 0.2 mM PN and 50 g/L lysine; FIG. 4D shows cell growth with 0.2 mM PN and 75 g/L lysine in LB; FIG. 4E shows cadaverine production by 75 g/L lysine; FIG. 4F shows PLP concentration in LB with 0.2 mM PN and 75 g/L lysine. All samples were measured after induction with 0.01 mM IPTG at 8 h or 48 h.* p-value<0.05.

FIG. 5 shows the whole-cell biotransformation of lysine to cadaverine in strains A, AJY, and APK with different concentrations of lysine (0.4 M, 0.8 M, and 1.2 M, respectively). Bar symbols: lysine consumption; Dot lines: cadaverine titer. * p-value<0.05.

FIG. 6 shows the protein expression of strains A, AJY, and APK after induction with 0.01 mM IPTG at 8 h. Lane 1: total protein in BL21(DE3); Lane 2: total protein in strain A; Lane 3: total protein in strain AJY; Lane 4: total protein in strain APK; Lane 5: protein marker. CadA, PdxY, and PdxK are indicated by arrows.

FIG. 7 shows the time course of cadaverine production by whole-cell transformation of strains A and APK, and cold-shock permeabilized cell of APK with 1.2 M lysine.

FIG. 8 shows the results of nuclear magnetic resonance spectroscopy for commercial cadaverine (upper) and distilled cadaverine (lower) by ¹H NMR spectra.

FIGS. 9A and 9B show the characterization of bio-based nylon 512 monomer salt. FIG. 9A: differential scanning calorimetry (DSC) curve; FIG. 9B: thermogravimetric analysis (TGA) curve.

FIG. 10 shows the results of comparing CadA activity of the JcadA-Wmut strain under the reaction condition of using PLP, PN or PL as a cofactor.

FIG. 11 shows the conversion rate from 1.2 M lysine to cadaverine and the cadaverine (DAP) production under the reaction condition of using the JcadA-Wmut strain as a whole-cell biocatalyst and PN or PL as the precursor.

FIG. 12 shows the conversion rate of the recycled JcadA-Wmut.

### DETAILED DESCRIPTION OF THE EMBODIMENTS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications.

It is further noted that, as used in this disclosure, the singular forms “a,” “an,” and “the” include plural referents, unless expressly and unequivocally limited to one referent. The term “or” is used interchangeably with the term “and/or” unless the context clearly indicates otherwise.

The present disclosure is directed to a recombinant microorganism for producing cadaverine, comprising a first nucleotide sequence encoding a first protein having lysine decarboxylase activity and a second nucleotide sequence encoding a second protein having pyridoxal kinase activity. By accompanying overexpression of proteins with activities of lysine decarboxylase and pyridoxal kinase, the recombinant microorganism provided in the present disclosure may be used as a whole-cell biocatalyst having improved ability to effectively produce cadaverine without adding external PLP.

As used herein, the term “microorganism” refers to a microscopic organism that comprises bacteria, archaea, viruses, or fungi. When employed in the descriptions herein, the term “microorganism” may be interpreted to encompass “bacteria.”

As used herein, the term “recombination” refers to artificially combining at least two separate sequence fragments. Generally, the term “recombinant” indicates that a nucleic acid, protein or microorganism contains genetic materials derived from multiple sources or is encoded by genetic materials derived from multiple sources, such as derived from two or more microorganisms of different strains or species.

In at least one embodiment, the recombinant microorganism of the present disclosure may be a recombinant microorganism of Escherichia, Klebsiella, Erwinia, Serratia, Providencia, Corynebacterium, or Brevibacterium. In some embodiments, the recombinant microorganism may be an E. coli strain. In some embodiments, the recombinant microorganism may be a recombinant microorganism of E. coli BL21 or E. coli BL21(DE3).

In at least one embodiment of the present disclosure, the first protein having lysine decarboxylase activity that may be expressed by the recombinant microorganism of the present disclosure is lysine decarboxylase 1 (CadA). In some embodiments, the first nucleotide sequence encoding the first protein having lysine decarboxylase activity has at least 80% identity to SEQ ID NO. 1.

As used herein, the term “lysine decarboxylase” refers to an enzyme which involves in the biotechnological production of cadaverine in an organism. Generally, there are two lysine decarboxylases involved in the bioconversion of lysine to cadaverine, namely CadA and lysine decarboxylase 2 (LdcC). CadA is an inducible enzyme that may be induced by oxygen starvation, excess supply of lysine and pH changes, whereas LdcC is a constitutive enzyme, which is independent of an external change in pH.

As used herein, the term “pyridoxal-5′-phosphate (PLP)” refers to the catalytically active form of vitamin B6. PLP plays a crucial role in cellular processes, such as serving as a versatile organic cofactor for the catalysis of diverse reactions on amino acids, oxoacids, and amine substrates. PLP may be synthesized by a super-salvage biosynthesis pathway in E. coli, which is a deoxyxylulose 5-phosphate (DXP)-dependent biosynthetic pathway. The genes pdxY, pdxK, and pdxH are involved in PLP production using the precursors, e.g., pyridoxine (PN), pyridoxal (PL), and pyridoxamine (PM).

In the present disclosure, PLP serves as a cofactor for the bioconversion of the lysine to the cadaverine. As used herein, the term “cofactor” refers to an agent that is necessary for an enzyme to be catalytically active, such as prosthetic groups and coenzymes, and that is not consumed in the process, meaning that it is found unchanged at the end of the enzymatic reaction.

As used herein, the term “pyridoxal kinase” refers to an enzyme that acts in the vitamin B6 salvage pathway to produce PLP. This enzyme belongs to the family of transferases, especially those transferring phosphorus-containing groups (phosphotransferases) with an alcohol group as an acceptor.

In at least one embodiment of the present disclosure, the second protein having pyridoxal kinase activity that may be expressed by the recombinant microorganism of the present disclosure is pyridoxal kinase selected from the group consisting of PdxH (pyridoxine/pyridoxamine 5′-phosphate oxidase, active in both salvage and de novo pathways), PdxK (biosynthesis pathway pyridoxalkinase), PdxY (salvage pathway pyridoxal kinase), and a combination thereof. In some embodiments, the second nucleotide sequence encoding the second protein having pyridoxal kinase activity has at least 80% identity to one of SEQ ID NOs. 2 to 4.

As used herein, the term “expression” refers to the biosynthesis of a gene product. For example, in the case of a structural gene, expression involves transcription of the structural gene into mRNA and the translation of mRNA into one or more polypeptides.

As used herein, the term “sequence identity” or, for example, comprising a “sequence 80% identical to,” as used herein, refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Additionally, various modifications may be made in the coding region, provided that they do not change the amino acid sequence of the polypeptide expressed from the coding region, due to codon degeneracy. That is to say, the present disclosure includes nucleotides and polypeptides having at least about 80%, about 85%, about 90%, about 95%, about 97%, about 98%, about 99% or about 100% sequence identity to any of the reference sequences described herein, typically where the polypeptide variant maintains at least one biological activity of the reference polypeptide.

In at least one embodiment, the recombinant microorganism of the present disclosure may comprise a first nucleotide sequence having at least 80%, at least 85%, at least 90%, at least 95%, at least 96%, at least 97%, at least 98% or at least 99% sequence identity to the nucleotide sequence of SEQ ID NO.1, and encoding lysine decarboxylase or a biologically active variant thereof.

In at least one embodiment, the recombinant microorganism of the present disclosure may comprise a second nucleotide sequence having at least 80%, at least 85%, at least 90%, at least 95%, at least 96%, at least 97%, at least 98% or at least 99% sequence identity to the nucleotide sequence of one of SEQ ID Nos. 2 to 4, and encoding pyridoxal kinase or a biologically active variant thereof.

In at least one embodiment of the present disclosure, the second nucleotide sequence further comprises a constitutive promoter for regulating the expression of the second protein. In some embodiments, the constitutive promoter may be promoter PlacI or a promoter belonging to J23100 series.

As used herein, the term “constitutive promoter” refers to a promoter that is in a permanent state of activity, allowing for gene expression in the absence of any activating biotic or abiotic regulatory factors; that is to say, the constitutive promoter can promote the transcription of a gene under the control thereof even in the absence of an inducer. The examples of a constitutive promoter suitable for the present disclosure include, but are not limited to, promoter J23100, J23101, J23102, J23103, J23104, J23105, J23106, J23107, J23108, J23109, J23110, J23111, J23112, J23113, J23114, J23115, J23116, J23117, J23118, J23119, and PlacI.

In at least one embodiment, the recombinant microorganism of the present disclosure comprises at least two expression vectors, and the first nucleotide sequence and the second nucleotide sequence are carried in the different expression vectors. For example, the recombinant microorganism comprises a first expression vector and a second expression vector, in which the first expression vector comprises the first nucleotide sequence encoding the first protein having lysine decarboxylase activity, and the second expression vector comprises the second nucleotide sequence encoding the second protein having pyridoxal kinase activity. In an alternative embodiment, the first nucleotide sequence and the second nucleotide sequence may be carried in the same expression vector for expressing the first protein having lysine decarboxylase activity and the second protein having pyridoxal kinase activity, respectively.

In at least one embodiment, the recombinant microorganism of the present disclosure is a cold-shocked microorganism, which has been subjected to a cold shock condition. In some embodiments, the recombinant microorganism of the present disclosure has been exposed to a cold temperature such as −80° C. to 25° C. (e.g., −80° C., −70° C., −50° C., −30° C., −20° C., −10° C., 0° C., 4° C., 10° C., 15° C., 20° C., or 25° C.).

The present disclosure is also directed to a method of producing cadaverine comprising culturing the above recombinant microorganism in a medium in absence of lysine to express the first protein having lysine decarboxylase activity and the second protein having pyridoxal kinase activity, mixing the cultured recombinant microorganism with lysine in a solution to perform bioconversion of lysine to cadaverine, and recovering cadaverine from the solution.

In at least one embodiment of the present disclosure, the medium for culturing the recombinant microorganism comprises an inducer capable of inducing the expression of the first protein having lysine decarboxylase activity, wherein the inducer may be in a concentration of from 5 μM to 50 μM (e.g., 5 μM, 7.5 μM, 10 μM, 15 μM, 20 μM, 25 μM, 30 μM, 35 μM, 40 μM, 45 μM, or 50 μM). The inducer suitable for the method of the present disclosure includes, but is not limited to, isopropyl β-d-1-thiogalactopyranoside (IPTG).

In at least one embodiment of the present disclosure, the method further comprises exposing the cultured recombinant microorganism to cold shock by storing the cultured microorganism at −80° C. to 25° C. (e.g., −80° C., −70° C., −50° C., −30° C., −20° C., −10° C., 0° C., 4° C., 10° C., 15° C., 20° C., or 25° C.) for 1 hour to 10 days (e.g., 1 hour, 8 hours, 12 hours, 16 hours, 20 hours, 1 day, 2 days, 3 days, 5 days, 7 days, 8 days, or 10 days). In some embodiments, the cold shock is performed before mixing the cultured recombinant microorganism with lysine.

In at least one embodiment of the present disclosure, the medium further comprises a substrate of the second protein having pyridoxal kinase activity, wherein the substrate may be in a concentration of from 0.1 μM to 0.5 mM (e.g., 0.1 μM, 0.5 μM, 1 μM, 2.5 μM, 5 μM, 10 μM, 20 μM, 30 μM, 40 μM, 50 μM, 60 μM, 70 μM, 80 μM, 90 μM, 0.1 mM, 0.15 mM, 0.2 mM, 0.25 mM, 0.3 mM, 0.35 mM, 0.4 mM, 0.45 mM, or 0.5 mM). In some embodiments, the substrate of the second protein having pyridoxal kinase activity is a substrate of pyridoxal kinase, such as pyridoxamine (PM), pyridoxine (PN), pyridoxal (PL), pyridoxamine-5′-phosphate (PMP), and pyridoxine-5′-phosphate (PNP).

In at least one embodiment of the present disclosure, the method further comprises separating the cultured recombinant microorganism from the medium, and adding the separated cultured recombinant microorganism into the solution to perform the mixing.

In at least one embodiment of the present disclosure, for mixing, the recombinant microorganism is formulated as an aqueous solution having a given OD₆₀₀ value. In some embodiments, the cultured recombinant microorganism is mixed in the solution after a concentration of the cultured recombinant microorganism represented by OD₆₀₀ in the medium achieves at least 0.5.

As used herein, the term “optical density (OD)” refers to a measure of cell growth and density of cells in the culture, and thus also refers to the concentration of the cells in a solution. OD₆₀₀ means the optical density measured at a wavelength of 600 nm. Unless otherwise indicated, “OD” as used herein refers to OD₆₀₀.

In at least one embodiment of the present disclosure, in the step of mixing, the concentration of the recombinant microorganism is represented by OD₆₀₀ from 0.5 to 6, and the concentration of lysine in the solution is from 0.1 M to 2.0 M. For example, the concentration of the recombinant microorganism may be OD₆₀₀ substantially equal to 0.5, 1, 2, 3, 4, 5 or 6, and the concentration of lysine in the solution may be 0.1 M, 0.2 M, 0.25 M, 0.3 M, 0.4 M, 0.5 M, 0.6 M, 0.7 M, 0.75 M, 0.8 M, 0.9 M, 1 M, 1.1 M, 1.2 M, 1.25 M, 1.3 M, 1.4 M, 1.5 M, 1.6 M, 1.7 M, 1.75 M, 1.8 M, 1.9 M, or 2 M. In some embodiments, the concentration of lysine in the solution may be from 10 g/L to 290 g/L. For example, the concentration of lysine in the solution may be 10 g/L, 15 g/L, 20 g/L, 25 g/L, 30 g/L, 40 g/L, 50 g/L, 60 g/L, 70 g/L, 75 g/L, 80 g/L, 90 g/L, 100 g/L, 110 g/L, 120 g/L, 125 g/L, 130 g/L, 140 g/L, 150 g/L, 160 g/L, 170 g/L, 175 g/L, 180 g/L, 190 g/L, 200 g/L, 210 g/L, 220 g/L, 225 g/L, 230 g/L, 240 g/L, 250 g/L, 260 g/L, 270 g/L, 275 g/L, 280 g/L, or 290 g/L.

Many examples have been used to illustrate the present disclosure. The examples below should not be taken as a limit to the scope of the present disclosure.

### EXAMPLES

### Materials and Methods

The materials and methods used in the following examples were described in detail below. The materials used in the present disclosure but unannotated herein are commercially available.

### (1) Materials

Luria-Bertani (LB) medium and agar were purchased from Oxoid (Basingstoke, UK). The antibiotics (e.g., kanamycin and chloramphenicol), dimethyl sulfoxide (DMSO) and isopropyl β-D-1-thiogalacto pyranoside (IPTG) were purchased from Sigma (St. Louis, USA). Cadaverine, dodecanedioic acid and diethyl ethoxymethylenemalonate (DEEMM) were purchased from Acros (Belgium). L-lysine monohydrochloride was purchased from Sangon Biotech (Taiwan). Acetonitrile (ACN), n-butanol, and methyl ethyl ketone (MEK) were purchased from ECHO (Taiwan). PCR primers were synthesized by IDT (USA), and DNA polymerase was purchased from Takara Biomedical Technology (USA). Restriction enzymes used herein were purchased from NEB (Taiwan).

### (2) Construction of Whole-Cell Biocatalysts

All DNA manipulations were performed according to standard protocols with modifications [12]. The lysine decarboxylase gene (cadA, ECK4125) (SEQ ID NO. 1) was amplified from genomic DNA of E. coli MG1655 (GenBank: NC_000913) and introduced into the vector of pSIT (plasmid #41684, purchased from Addgene) to generate pSIT-P_{T7}-CadA (SEQ ID NO. 7). Further, the genes of pdxH (ECK1634) (SEQ ID NO. 2), pdxK (ECK2413) (SEQ ID NO. 3) and pdxY (ECK1632) (SEQ ID NO. 4) were amplified from E. coli MG1655 and ligated into pSUJ or pSUP to generate the plasmids of pJH, pPH, pJK, pPK, pJY and pPY. The constructed plasmids were confirmed by enzymatic digestion and DNA sequencing, which were performed by Genomics (Taiwan).

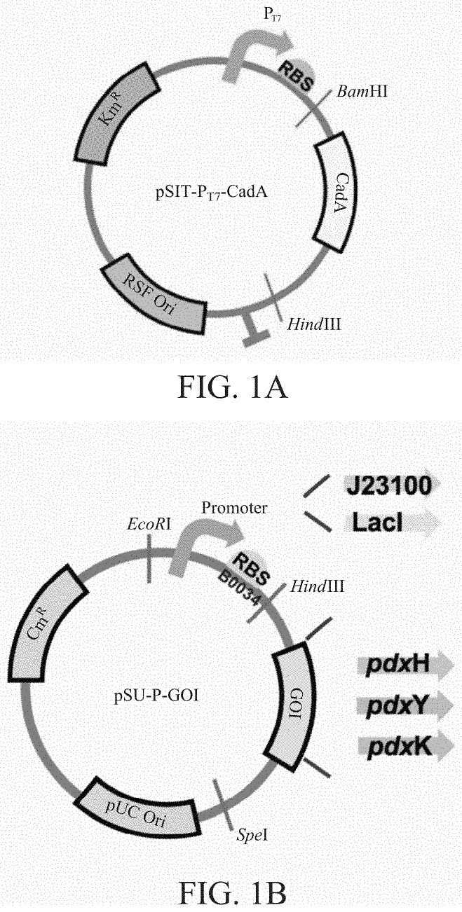

The construction maps of pSIT-P_{T7}-CadA and pSU-P-GOI were illustrated in FIGS. 1A and 1B, respectively. In the pSU-P-GOI, the promoter may be J23100 (SEQ ID NO. 5), which was originated from pSUJ, or may be PlacI (SEQ ID NO. 6), which was originated from pSUP. Further, the gene of interest (GOI) may be pdxH, pdxK, or pdxY; therefore, the plasmids of pJH (J23100+pdxH), pPH (PlacI+pdxH), pJK (J23100+pdxK), pPK (PlacI+pdxK), pJY (J23100+pdxY) and pPY (PlacI+pdxY) comprising different promoters and different GOIs were obtained.

E. coli DH5α, E. coli W3110, E. coli BL21, and E. coli BL21(DE3) were used as host strains. The constructed plasmid, pSIT-P_{T7}-CadA, pJH, pPH, pJK, pPK, pJY, or pPY, was transformed into the host strain competent cells to produce the recombinant E. coli cells as whole-cell biocatalysts. The plasmids, primers and Escherichia coli strains used herein were summarized in Tables 1 to 3 below, respectively.

TABLE 1
Plasmids used herein

| Plasmids | Description |
|---|---|
| pSIT | RSF ori, T7 promoter, Km^{R} |
| pSIT-P_{T7}-CadA | RSF ori, T7 promoter, CadA, Km^{R} |
| (SEQ ID NO. 7) | |
| pSU | pUC ori, sfGFP, Cm^{R} |
| (SEQ ID NO. 8) | |
| pSUJ | pUC ori, J23100 promoter, sfGFP, Cm^{R} |
| pSUP | pUC ori, PlacI promoter, sfGFP, Cm^{R} |
| pJH | pSUJ, sfGFP:: pdxH |
| pPH | pSUP, sfGFP:: pdxH |
| pJK | pSUJ, sfGFP:: pdxK |
| pPK | pSUP, sfGFP:: pdxK |
| (SEQ ID NO. 9) | |
| pJY | pSUJ, sfGFP:: pdxY |
| (SEQ ID NO. 10) | |
| pPY | pSUP, sfGFP:: pdxY |

sfGFP: superfolder green fluorescent protein

TABLE 2
Primers used herein

| Primers | Sequence | SEQ ID NO |
|---|---|---|
| JH/PH-F | 5’-GCAAGCTTATGTCTGATAACGACGAATTGC-3’ | 11 |
| JH/PH-R | 5’-CTACTAGTTCAGGGTGCAAGACGATCAATC-3’ | 12 |
| JY/PY-F | 5’-GCGCTTATGATGAAAAATATTCTCGCTATCC-3’ | 13 |
| JY/PY-R | 5’-CTACTAGTTCAGAGCTTTGTTGCGCTGAAGT-3’ | 14 |
| JK/PK-F | 5’-GCGCTTATGAGTAGTTTGTTGTTGTTTAACG-3’ | 15 |
| JK/PK-R | 5’-ACTAGTTTATGCTTCCGCCAGCGGCGGCAA-3’ | 16 |
| pSIT-P_{T7}-CadA-F | 5’-GCGGATCCATGAACGTTATTGCAATATTGAAT-3’ | 17 |
| pSIT-P_{T7}-CadA-R | 5’-GCAAGCTTTTTTTTGCTTTCTTCTTTCAATAC-3’ | 18 |

TABLE 3
Escherichia coli strains used herein

| Strains | Description |
|---|---|
| DH5α | F⁻ endA1 glnV44 thi-1 recA1 relA1 gyrA96 deoR nupG |
| | purB20 φ80d lacZΔM15 Δ(lacZYA-argF) U169, |
| | hsdR17(r_{K}⁻m_{K}⁺), λ⁻ |
| W3110 | F⁻ λ⁻ IN(rrnD-rrnE)1, rph-1 |
| BL21 | F⁻ ompT gal dcm lon hsdS_{B} (r_{B}⁻m_{B}⁻) [malB+]_{K-12} (λ^{S}) |
| BL21(DE3) | F⁻ ompT gal dcm lon hsdS_{B} (r_{B}⁻m_{B}⁻) λ(DE3 [lacI |
| | lacUV5-T7p07 ind1 sam7 nin5]) [malB⁺]_{K-12}(λ^{S}) |
| A | BL21(DE3) harboring the pSIT-P_{T7}-CadA |
| AJY | BL21(DE3) harboring the pSIT-P_{T7}-CadA and pJY |
| APK | BL21(DE3) harboring the pSIT-P_{T7}-CadA and pPK |

### (3) Culture Condition

Recombinant E. coli cells harboring the appropriate plasmids were inoculated into 50 mL of LB medium supplemented with kanamycin (50 μg/mL) or chloramphenicol (25 μg/mL) in 250-mL baffle shaking flasks at 37° C. A precursor, pyridoxine (PN) or pyridoxal (PL), at 0.2 mM was added into the medium for PLP biosynthesis. For the strain harboring pSIT-P_{T7}-CadA upon reaching an OD₆₀₀ of 0.5, the cells were induced by 0.01 mM IPTG, and transferred to 30° C. incubator for protein expression. Cell density was determined at appropriate intervals by measuring the turbidity of the culture medium at OD₆₀₀ using a spectrophotometer (Molecular Devices, USA). Measurement of the dry cell biomass (g/L) was performed at 110° C. for 20 min in an Infrared Moisture Analyzer (FD-660, Kett Klectric Laboratory, Japan) until the weight was constant. The calibration between OD₆₀₀ and dry cell weight (g/L) were calculated by the equation Y (g/L)=0.30× (OD₆₀₀) for BL21, and Y (g/L)=0.33×(OD₆₀₀) for W3110.

For in vivo whole-cell biotransformation of lysine to cadaverine, 50 g/L or 75 g/L L-lysine was added into the cultured E. coli strain when IPTG was added for induction, and then such reaction mixture was transferred to a 30° C. incubator. The concentrations of L-lysine and cadaverine in the reaction mixture were determined at 8, 20, 30 and 48 hours after induction. The mixture was centrifuged at 8,000×g for 10 min to alleviate the cells, and the supernatant was analyzed via high pressure liquid chromatography (HPLC).

For in vitro whole-cell biotransformation of lysine to cadaverine, after the recombinant E. coli cells were incubated with induction of 0.01 mM IPTG at 30° C. for 16 hours, the cells were harvested by centrifugation at 6,000×g for 10 min at 4° C., and washed twice with a 0.9% saline solution. For the cold shock treatment, the cells were washed twice, followed by removing the liquid and storing at −20° C. for 2 days. The reaction mixture for biotransformation of lysine to cadaverine contained 0.4 M (60 g/L), 0.8 M (120 g/L) or 1.2 M (180 g/L) L-lysine and 3.0 g-DCW/L of whole-cell biocatalyst in a 0.9% saline solution with 5 mL at 37° C. The reaction was stopped after 6 h by heating at 100° C. for 10 min. The concentrations of L-lysine and cadaverine in the reaction mixture were determined by HPLC.

### (4) Analysis of Lysine and Cadaverine by HPLC

Since diamine is a weak signal, derivatives were obtained by the reaction of 340 μL of 0.05 M borate buffer (pH 9), 240 μL of 100% methanol, 6 μL of sample and 12 μL of 200 mM DEEMM. The samples were heated at 70° C. for 2 h to allow complete degradation of excess DEEMM and derivatization [13]. After derivatization with DEEMM, analyses were performed by HPLC (Hitachi, Japan) consisting of a quaternary-pump, an inline degasser, an autosampler, and a column thermostat. Chromatographic separation was carried out by reverse-phase chromatography on a C18 column (YMC-C18 column, 4.6×250 mm, 5 μm particle size), maintained at 35° C. Mobile phase A was composed of 100% acetonitrile, and mobile phase B was made up of 25 mM aqueous sodium acetate buffer (pH 4.8). The flow rate of 0.8 mL/min was used, with the following gradient program: 0-2 min, 20-25% mobile phase A; 2-32 min, 25-60% mobile phase A; and 32-40 min, 60-20% mobile phase A. Detection was carried out at 284 nm.

### (5) Analytical Method of PLP

For analyzing the level of PLP in the cells, 10 mg (dry cell weight) cell pellets were resuspended in 1.5 mL sodium chloride saline and disrupted by high pressure homogenizer at 30 kPsi (One-Shot, UK). Then, proteins were precipitated by adding chilled 100% trichloroacetic acid (TCA) at 1/10 of the sample volume. The samples were vortexed for 1 min and incubated for 15 min on ice, and then were centrifuged for 10 min at 12,000×g at 4° C. The supernatant was cleared by 0.2 μm filtration. The quantification of PLP was conducted by HPLC, following modifications of the procedure described by Kimura et al. [14]. The stationary phase was a reverse-phase column (YMC-C18 column, 4.6×250 mm, 5 μm in particle size). The elution buffer was 0.1 M potassium phosphate monobasic (pH 6.6) for elution time, 0 to 20 min. The flow rate was 0.8 mL/min, and the injection volume was 50 μL. The temperature of the column was equilibrated at 35° C., and PLP was measured at wavelength of 340 nm.

### (6) Purification and Characterization of Cadaverine

Solvent extraction was conducted to extract cadaverine in the fermentation broth as described by Kim et al. with some modifications [15]. An appropriate amount of NaOH was added to the supernatant in a glass reactor, and the pH was adjusted to 14. The same volume of MEK was added to the reactor and mixed with the pH-adjusted supernatant using a magnetic heat stirrer at 300 rpm, 55° C. for 2 h. The mixture was then transferred into separation funnels for phase separation. The organic phase was collected in a round-bottom flask for further procedures, and the aqueous phase was treated again with the same extraction procedure as described above. The collected organic phase from the first and second extractions was concentrated using a rotary evaporator at 60° C. and 175 rpm. Fractional distillation was carried out using a laboratory distillation apparatus at 25-120 mbar and 120-135° C. to yield cadaverine.

The purity of distilled cadaverine was determined by HPLC as described above. Then, cadaverine was dissolved in dimethyl sulfoxide (DMSO) for proton nuclear magnetic resonance (1H NMR, 600 MHz, Bruker) analysis. Chemical shifts were expressed in parts per million (ppm) scales.

### (7) Synthesis of Bio-Based PA512 Monomer Salt

The crystallization was carried out with n-butanol, of which 6.77 g dodecanedioic acid was added into the cadaverine-butanol mixtures (3.0 g of cadaverine was dissolved in 8.64 mL of absolute n-butanol) to reach the molar ratio of 1:1, followed by mixing thoroughly until the solution became neutral. The solution was heated up to 45° C. and agitated at 500 rpm for 30 min. The white crystal salt was obtained and followed by washing with lots of ethanol to remove the remained cadaverine and dodecanedioic acid. The purified salt was dried at vacuum drying oven at 70° C. for 12 h. The dried salt was refined in ethanol for three times to get the final product.

### (8) Characterization of PA512 Monomer Salt

Melting temperature (Tm) was measured by differential scanning calorimetry (DSC, 6000, PerkinElmer, USA). The PA512 monomer sample was melted gradually at 20° C./min from 30° C. to 250° C. in N2 atmosphere. The degradation temperature (Td) was determined by thermogravimetric analysis (TGA, 4000, PerkinElmer, USA). The PA512 monomer sample was heated from 30° C. to 500° C. at 10° C./min under N2.

### Example 1: Effect of Super-Salvage Pathway Genes on Cell Growth

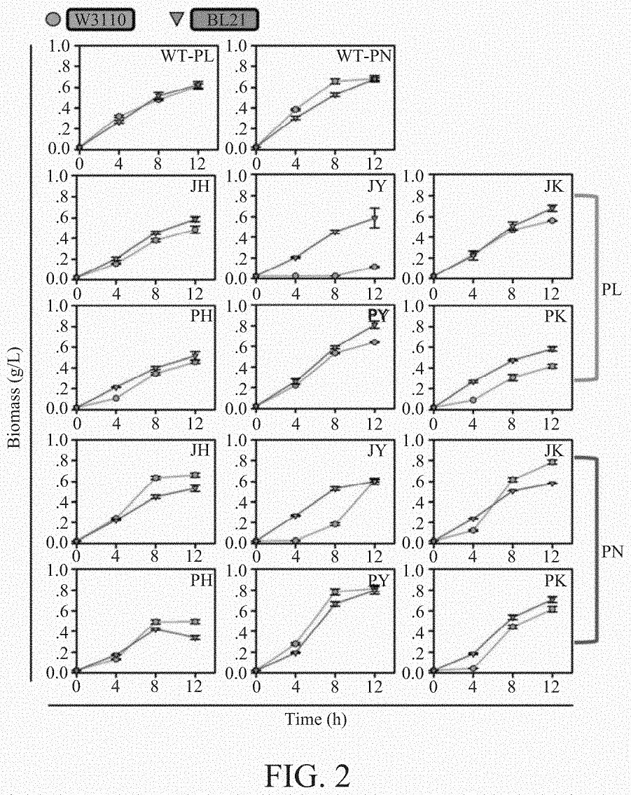

The profiles of cell growth for recombinant E. coli W3110 and BL21 strains harboring the plasmids pJH, pJK, pJY, pPH, pPK, and pPY were shown in FIG. 2. W3110 grew slowly in the precursor PL than that in the precursor PN. Moreover, the lowest biomass was obtained in W3110 when harboring plasmid pJY in the presence of PL. The lag-phase of W3110 with pJY plasmid was extended, and thus the biomass with PL was only 0.05 g/L at 6 h, while the biomass with PN was 0.2 g/L at 6 h. Further, a higher biomass was obtained in W3110 when harboring pJH and pPH plasmids with PN. The engineered BL21 strains, which harbor different plasmids, grew quickly in the first 6 h compared to W3110. In addition, BL21 harboring the pJY plasmid did not show inhibition of cell growth compared with BL21 wild-type. Among different plasmids in BL21, cell growth gradually reduced after 8 h with pPH and PN as the precursor, while the biomass of BL21 harboring pPY might reach to 0.9 of OD. These results of biomass implied the different amount of PLP accumulation in the recombinant strains, and the detail of protein expression and PLP amount were discussed below.

### Example 2: Optimal Production of Intracellular PLP

The protein expression level of recombinant E. coli DH5α, W3110, and BL21 strains harboring the plasmids pJH, pJK, pJY, pPH, pPK, and pPY were measured. The calculated molecular weights of PdxH, PdxY, and PdxK were 25.5, 31.3, and 30.9 kDa, respectively.

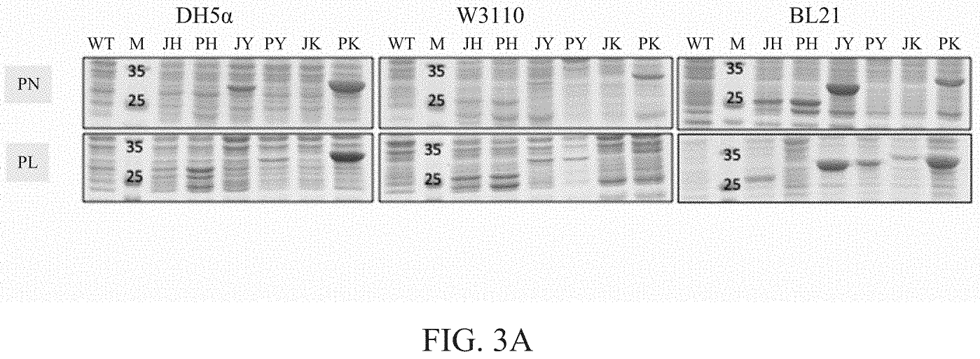

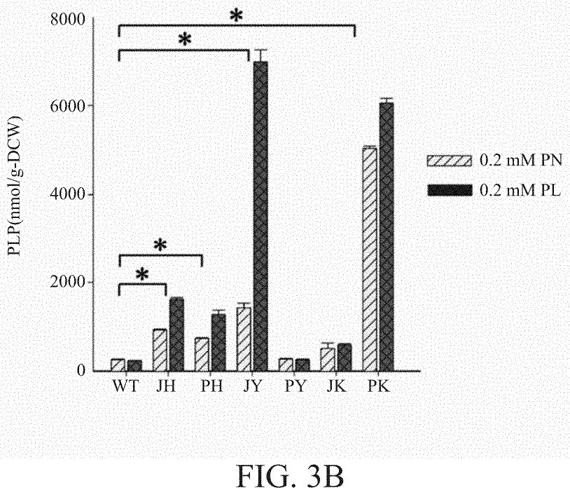

As shown in FIG. 3A, the protein expression levels in the three strains were significantly different from each other. Interestingly, all of the strains exhibited an unexpected increasing pattern of protein expression when cultured with 0.2 mM PL, which was relatively obvious in strain BL21. All three genes were highly overexpressed in strain BL21, especially pdxY under the J23100 promoter and pdxK under the PlacI promoter. Further, pdxK expression under the PlacI promoter was clearly observed in W3110 strain.

In addition, by analyzing the level of PLP in the cells, it was observed that in the presence of 0.2 mM PN or PL, DH5a and W3110 showed similar trends with increase in intracellular PLP observed for all three genes (i.e., pdxK, pdxH and pdxY). Specifically, a 4.5-fold and 29.3-fold increase in the cellular PLP level was observed in the pJH-harboring strain compared to the W3110 wild-type strain using PN and PL as the precursor, respectively.

Also, referring to FIG. 3B, pdxK could assist to supply intracellular PLP in strain BL21, with the intracellular concentration increase to 6053 nmol/g-DCW. The intracellular PLP level in strain BL21 harboring the pdxY gene under the J23100 promoter using PN as a precursor reached 7008 nmol/g-DCW, which was the highest accumulation observed among all tested strains. By contrast, no intracellular PLP could be detected in wild-type BL21, since the PLP produced by the strain itself is only sufficient to maintain cell growth and metabolism without providing any surplus.

From the above, among all E. coli strains tested, the BL21 strain carrying pJY showed the relatively high intracellular PLP production with PN as a precursor, and pPK also had a superior effect on intracellular PLP enhancement.

### Example 3: Enhancement of Cadaverine Production by Intracellular PLP

Although some microbes, including E. coli, have the capability to synthesize the essential cofactor of PLP, the amount produced may be generally insufficient for the activity of heterogenous enzymes, leading to a poor catalytic effect. Therefore, in this example, pSIT-P_{T7}-CadA in combination with pJY or pPK were co-transformed into BL21(DE3) cells to evaluate the conversion of L-lysine to cadaverine based on the intracellular PLP results as mentioned above.

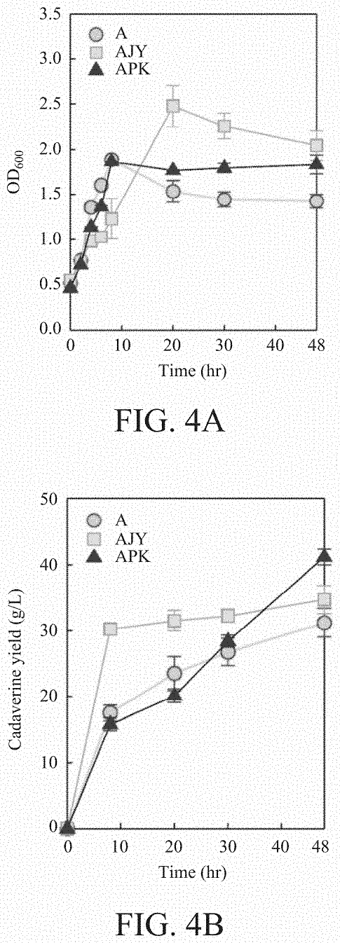

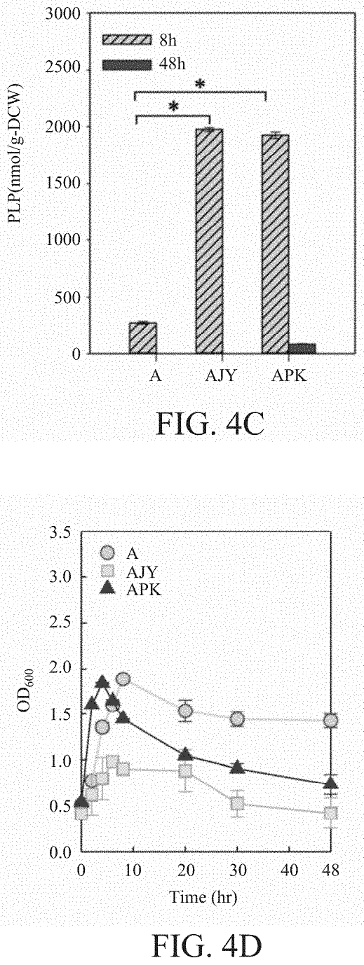

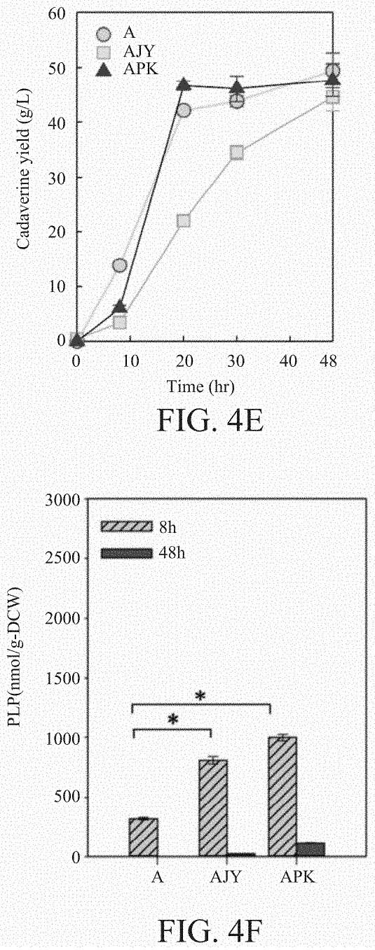

As shown in FIG. 4A, the biomass in MY was the highest (i.e., 0.744 g/L; OD₆₀₀ was 2.48) with 50 g/L lysine at 20 h, and cadaverine production was increased quickly in the first 6 h and remained stable afterwards. The biomass of APK and A strains was from 0.45 g/L to 0.57 g/L. The cadaverine production was ranking from 41.2 g/L in APK, 34.7 g/L in MY and 31.2 g/L in A at 48 h (FIG. 4B). These results were consistent with PLP production and accumulation that a significant increase in intracellular PLP production was observed in strain AJY (i.e., 1975 nmol/g-DCW) and APK (i.e., 1926 nmol/g-DCW), while the intracellular PLP in strain A was only 272 nmol/g-DCW at 8 h (FIG. 4C and Table 4). The intracellular PLP was exhausted (i.e., zero) in all the 3 strains at 48 h, indicating that higher L-lysine conversion required supplementary PLP to maintain CadA activity in vivo. These results demonstrated that the higher lysine conversion resulted from a higher level of in vivo PLP. That is to say, a strain harboring the pdxY or pdxK gene could promote the intracellular PLP pool, resulting in the enhanced activity of CadA.

The biomass of A strain was similar in 50 g/L and 75 g/L L-lysine supplement (FIG. 4D), while the cadaverine concentration increased to 47.9 g/L at 48 h when extra L-lysine was added into the culture (FIG. 4E). The APK strain showed a sharp decline in biomass with 75 g/L L-lysine after 4 h (FIG. 4D) due to the rapid and higher cadaverine production of 46.7 g/L (FIG. 4E), while the AJK strain grew slowly in 75 g/L L-lysine. The results implied that increased decarboxylase activity can be achieved from the pdxK gene with the addition of PN.

In the reaction with 75 g/L lysine, strains AJY and APK entered the death phase in the logarithmic growth phase (FIG. 4D). Therefore, it became an in vitro biotransformation reaction. The PLP production of strains AJY and APK were 809 and 996 nmol/g-DCW, which were almost three-fold higher than that of strain A (317 nmol/g-DCW) at 8 h (FIG. 4F).

TABLE 4
Comparison of intracellular PLP concentrations and cadaverine
concentrations

| | PLP | | | PLP | | |
|---|---|---|---|---|---|---|
| | (nmol/ | | Cadaverine | (nmol/ | | Cadaverine |
| | g-DCW) | OD₆₀₀ | (g/L) | g-DCW) | OD₆₀₀ | (g/L) |
| Strains | 50 g/L lysine | | | 75 g/L lysine | | |
| 8 h | | | | | | |
| A | 272 | 1.89 | 17.57 | 317 | 1.89 | 13.86 |
| AJY | 1975 | 1.23 | 30.23 | 809 | 0.90 | 3.45 |
| APK | 1926 | 1.87 | 15.83 | 996 | 1.45 | 6.03 |
| 48 h | | | | | | |
| A | ND | 1.43 | 31.19 | ND | 1.43 | 49.42 |
| AJY | ND | 2.04 | 34.70 | 21.9 | 0.42 | 44.62 |
| APK | 79 | 1.83 | 41.19 | 109 | 0.73 | 47.67 |

*ND: non-determined.

### Example 4: Enhancement of Lysine Conversion by Whole-Cell Biotransformation

In this example, the effects of the pdxY and pdxK genes on increasing the intracellular PLP pool to enhance the CadA activity were examined.

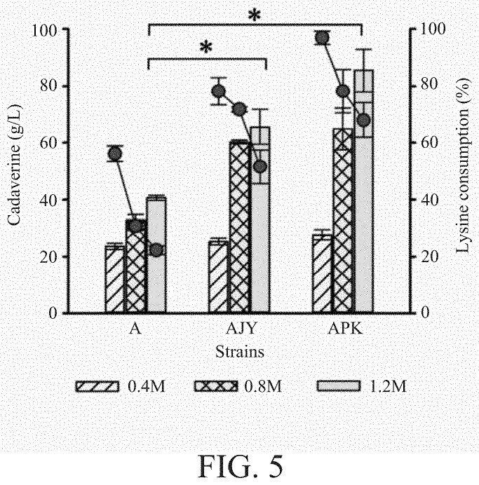

Referring to FIG. 5, strain A showed a relatively high conversion rate of 56.2% at 0.4 M lysine, which decreased to 30.8% with 0.8 M lysine and to 22.2% with 1.2 M L-lysine. Therefore, the highest cadaverine produced by strain A was approximately 27.8 g/L by in vitro biotransformation.

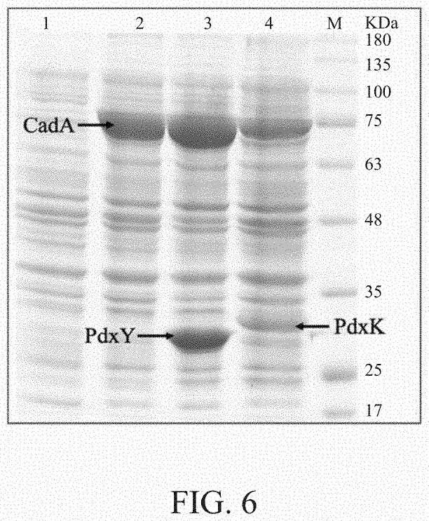

However, strain AJY showed significant increment, and obtained 78.43%, 71.6%, and 51.63% conversion with 0.4 M, 0.8 M, and 1.2 M L-lysine, respectively. All strains showed comparable protein expression levels (FIG. 6). On the other hand, strain APK showed a significant increase in lysine conversion up to 97% with 0.4 M, 78.2% with 0.8 M and 68% with 1.2 M L-lysine (FIG. 5). The highest cadaverine was up to 83.2 g/L in APK with 1.2 M L-lysine which was 2-folds higher as compared with strain A. It should be noted that the lysine conversion was determined by total PLP concentration with high cell density for whole cell biotransformation.

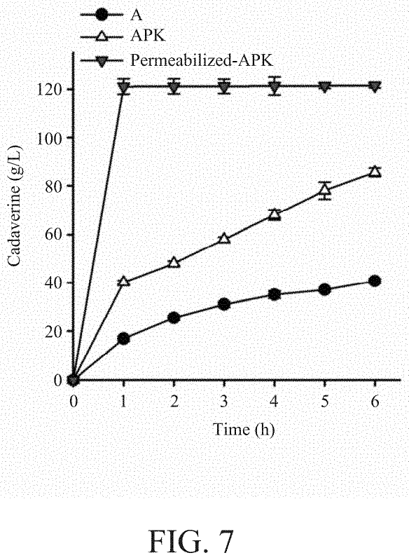

Furthermore, an efficient and eco-friendly system for the successful conversion of high-level L-lysine to cadaverine in a short time was developed by treating the biocatalysts by cold shock. As shown in FIG. 7, APK with cold treatment successfully converted 1.2 M L-lysine into cadaverine within one hour, and achieved a cadaverine titer, molar yield and productivity of 121 g/L, 99.2% and 121 g/L/h.

The results of this example were summarized in Table 5 below.

TABLE 5
Summary of cadaverine titer, productivity and specific
productivity from different recombinant strains

| | | Cada- | Produc- | Specific | Reaction |
|---|---|---|---|---|---|
| Amplified | | verine | tivity | productivity | time |
| genes | Strategy | (g/L) | (g/L/m) | (g/g-DCW/h) | (h) |
| CadA with | Regeneration | 63.2 | 10.5 | 3.5 | 6 |
| pdxY | of PLP | | | | |
| CadA with | Regeneration | 83.2 | 13.9 | 4.6 | 6 |
| pdxK | of PLP | | | | |
| CadA with | Cold-treated, | 121 | 121 | 40.3 | 1 |
| pdxK | regeneration | | | | |
| | of PLP | | | | |

These results indicated that pdxK and pdxY clearly increase the PLP pool. Thus, the coupling of CadA and PLP-related enzymes such as those encoded by pdxK and pdxY in vivo was successfully achieved in recombinant E. coli, enabling independent lysine conversion owing to intrinsic PLP production.

### Example 5: Characterization of Cadaverine and PA512 Monomer Salt

After solvent extraction, concentration, and distillation, bio-based cadaverine with high purity was determined by HPLC (>99%). Furthermore, nuclear magnetic resonance (NMR) was conducted to compare with commercial cadaverine and the distillated product.

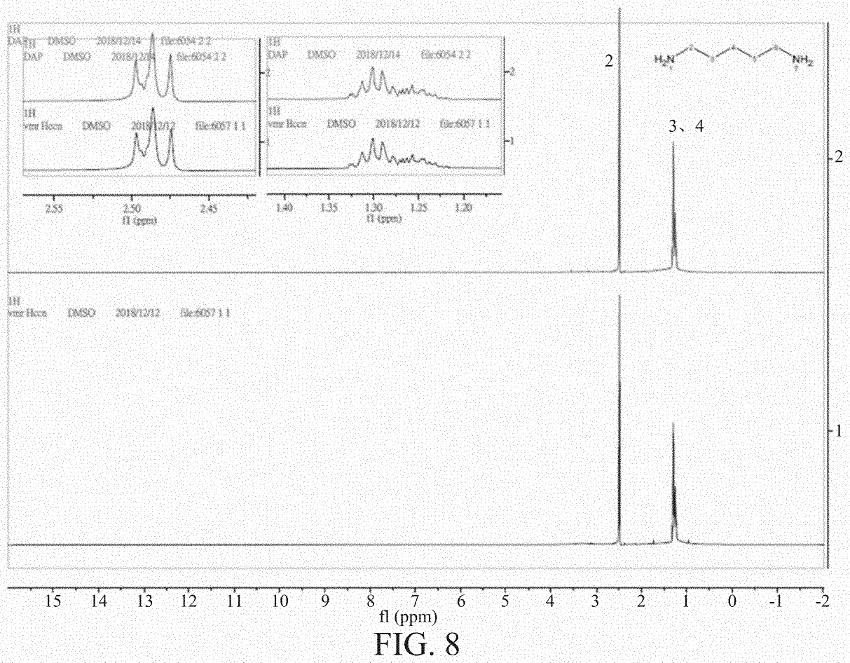

As shown in FIG. 8, the upper was commercial cadaverine, while the lower was cadaverine produced by the recombinant E. coli BL21(DE3), which major peaks corresponding to cadaverine were detected.

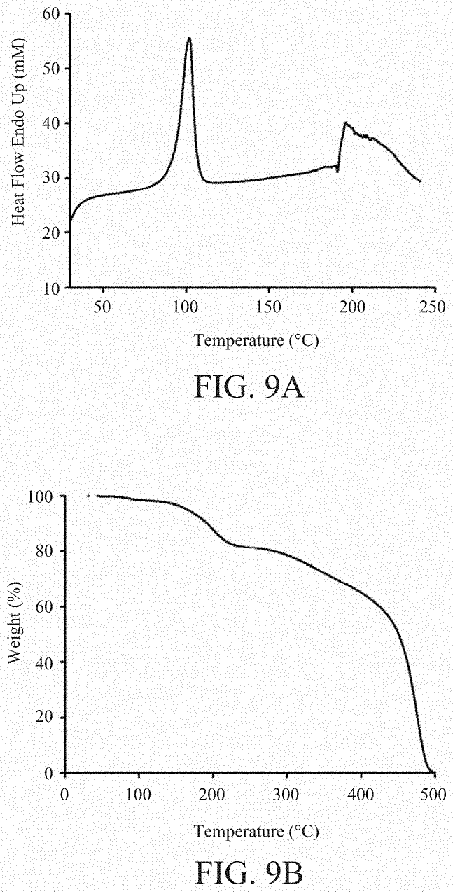

Further, PA512 monomer salt was synthesized from the purified cadaverine and dodecanedioic acid. To characterize the chemical properties of the synthesized PA512 monomer salt, differential scanning calorimetry (DSC) analysis and thermogravimetric analysis (TGA) were conducted. The onset temperature of the endothermic peak was 102±1.5° C. (FIG. 9A). FIG. 9B showed three steps by weight loss of bio-based PA512 monomer salt during heating. Two different types of degradation mechanism were observed, namely dehydration and decomposition. The first stage of degradation was the loss of adsorbed water. The endothermic reaction at the end of DSC analysis with an obvious weight loss was detected in the TG curve at 196° C. Finally, the maximum decomposition rate was defined by one-order derivative, which was at 464° C., indicating that the synthesized PA512 was a high heat-resistant material.

### Example 6: Construction of E. coli Stain JcadA-Wmut

In this example, a recombinant E. coli strain, JcadA-Wmut, was constructed by transforming pSU-J23100-CadA (SEQ ID NO. 19) into strain W3110 and culturing the strain in LB medium containing pyridoxal (PL) in a concentration of 10 μM to 150 μM. After culture overnight, the strain was inoculated in a fresh medium containing high concentration of PL, and such culture was subcultured 10 times. Subsequently, the strain was inoculated uniformly on an LB agarose plate (pH 5.0) containing 20 g/L lysine and 100 μM PL, and thus a single colony was selected for CadA activity assay.

As a result, the E. coli strain JcadA-Wmut has the capability to overexpress PLP and CadA, and exhibits excellent CadA activity. JcadA-Wmut has been deposited under Budapest Treaty at DSMZ-DEUTSCHE SAMMLUNG VON MIKROORGANISMEN UND ZELLKULTUREN GmbH (Inhoffenstr. 7 B, D-38124 Braunschweig, Germany) on Jan. 11, 2021 and has been given the DSMZ Accession No. DSM 33754 by the International Depositary Authority. This biological material was subjected to the viability test and passed.

### Example 7: Comparison of CadA Activity of JcadA-Wmut

In order to compare the CadA activity with different cofactors, the JcadA-Wmut cells cultured overnight were centrifuged at 8,000×g for 5 min, and then the pellet was collected and washed twice with pure water. After then, the cells were added into a solution containing 1.2 M lysine and different cofactors, such as PLP, PN or PL, for the whole-cell bioconversion at 37° C. under 200 rpm shaking conditions.

The activity of lysine decarboxylase CadA was measured with bromophenol purple (BP) assay as follows:

First, the bacterial mass was quantified to OD₆₀₀=5, and then high-pressure disruption (30 Kpsi) was performed followed by centrifugation at 12,000×g for 5 min to obtain a soluble protein sample containing lysine decarboxylase. The amount of diaminopentane produced was increased by the catalytic reaction of lysine decarboxylase. BP coloring agent can be detected at a wavelength of 595 nm, in which the BP agent will present purple under alkaline condition and present yellow under acidic condition. The enzyme activity can be calculated by conversion between the wavelength difference of 595 nm (Δ OD₅₉₅) and the calibration curve of diaminopentane quantified by HPLC, and it was found that when the initial lysine concentration was 40 mM, the best correlation between diaminopentane and HPLC result could be obtained. The reaction conditions listed in Table 6 below were used to measure the activity of lysine decarboxylase, in which the reaction was quenched by adding 3% trichloroacetic acid (TCA).

TABLE 6
Reaction conditions of the assay for measuring
the activity of lysine decarboxylase

| Item | Concentration | Volume (μL) |
|---|---|---|
| Lysine | 40 mM | 40 |
| PLP | 0.01 mM | 10* |
| BP coloring agent | 0.2 g/L | 25 |
| Sample to be | OD₆₀₀ = 5 | 25 |
| tested | | |
| Sodium acetate | 0.5M | 400 |
| buffer, pH = 6 | | |
| Total volume | | 500 μL |

*PLP can be omitted when the activity of CadA of a mutant strain is measured.

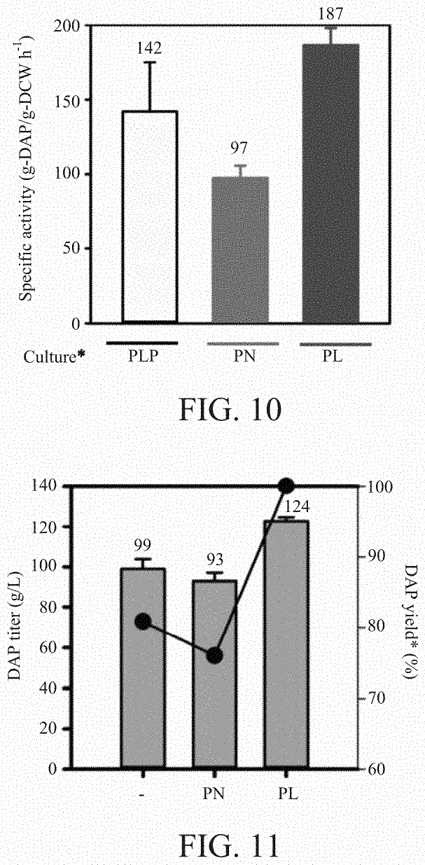

The results were shown in FIG. 10, indicating that JcadA-Wmut exhibited higher CadA activity when PL, but not PLP, was used as a cofactor in the reaction. Further, as shown in FIG. 11, by using JcadA-Wmut as a whole-cell biocatalyst and PL as a precursor, 1.2 M lysine could be completely converted to cadaverine without adding external PLP, and 124 g/L of cadaverine via up to 95% conversion from lysine could be achieved.

### Example 8: Catalytic Effect of the Recycled JcadA-Wmut as Whole-Cell Biocatalyst

The cells in the lysine and whole-cell biocatalyst solution used in Example 7 were centrifuged at 12,000×g for 1 min and recovered. The recovered cells were added into another solution containing 2 M lysine and the new JcadA-Wmut cells were further fed for bioconversion. The fed amount was OD₆₀₀=1, 2, 3, or 5, and JcadA-Wmut cells were recycled twice. Each reaction time was 4 hours, and after the reaction, the concentrations of lysine and cadaverine in the solution were measured by HPLC.

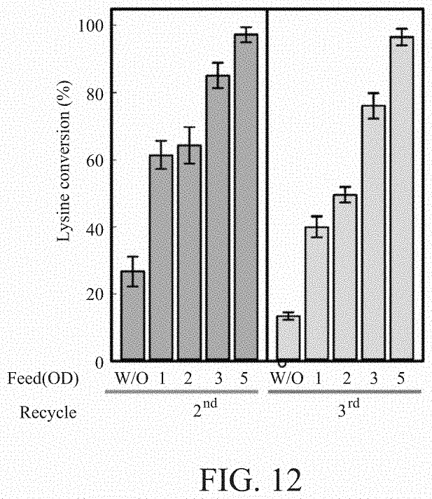

The results were shown in FIG. 12, indicating that JcadA-Wmut could be recycled twice under the condition that the fed amount was OD₆₀₀=5, and still exhibited the conversion rate of lysine of more than 95%.

From the above, the present disclosure provides an engineered E. coli system that has dual plasmids to co-expressing a PLP-dependent protein and CadA. By synthesizing PLP in vivo, the E. coli system can accelerate cadaverine production spontaneously in absence of external PLP and achieve 41.2 g/L of cadaverine via 100% conversion from 50 g/L lysine; also, the higher cadaverine productivity might be reached via using cold-shock permeabilized cells. Further, the purification process is enhanced by using MEK as an extraction solvent, which results in the production of polymer-grade bio-based cadaverine. Crystallization of purified cadaverine with dodecanoic acid results in bio-based PA512 monomer salt. The recombinant E. coli strains of the present disclosure and purification strategies of these strains can enhance the production of bio-based cadaverine, which are useful to develop a more economic and feasible process for sustainable production of bio-based nylon, demonstrating an immense potential in future industrial application.

While some of the embodiments of the present disclosure have been described in detail above, it is, however, possible for those of ordinary skill in the art to make various modifications and changes to the particular embodiments shown without substantially departing from the teaching and advantages of the present disclosure. Such modifications and changes are encompassed in the scope of the present disclosure as set forth in the appended claims.

### REFERENCES

[1] R. Muthuraj, M. Hajee, A. R. Horrocks, B. K. Kandola, Biopolymer blends from hardwood lignin and bio-polyamides: Compatibility and miscibility, Int. J. Biol. Macromol. 132 (2019) 439-450.
[2] N. Prakash, S. Arungalai Vendan, Biodegradable polymer based ternary blends for removal of trace metals from simulated industrial wastewater, Int. J. Biol. Macromol. 83 (2016) 198-208.
[3] L. Francesca, T. Luigi, M. K José, P. Debora, Bio- and fossil-based polymeric blends and nanocomposites for packaging: structure-property relationship, Materials 12 (2019) 471.
[4] J. H. Kim, H. M. Seo, G. Sathiyanarayanan, S. K. Bhatia, H. S. Song, J. Kim, J. M. Jeon, J. J. Yoon, Y. G. Kim, K Park, Y. H. Yang, Development of a continuous L-lysine bioconversion system for cadaverine production. J. Ind. Eng. Chem. 46 (2017) 44-48.
[5] Z. G. Qian, X. X. Xia, S. Y. Lee, Metabolic engineering of Escherichia coli for the production of cadaverine: a five carbon diamine, Biotechnol. Bioeng. 108 (2011) 93-103.
[6] W. Ma, W. Cao, H. Zhang, K. Chen, Y. Li, P. Ouyang, Enhanced cadaverine production from L-lysine using recombinant Escherichia coli co-overexpressing CadA and CadB, Biotechnol. Lett. 37 (2015) 799-806.
[7] Y. H. Oh, K. H. Kang, M. J. Kwon, J. W. Choi, J. C. Joo, S. H. Lee, Y. H. Yang, B. K. Song, I. K. Kim, K. H. Yoon, K. Park, S. J. Park, Development of engineered Escherichia coli whole-cell biocatalysts for high-level conversion of L-lysine into cadaverine, J. Ind. Microbiol. Biotechnol. 42 (2015) 1481-1491.
[8] H. J. Kim, Y. H. Kim, J. H. Shin, S. K. Bhatia, G. Sathiyanarayanan, H. M. Seo, K. Y. Choi, Y. H. Yang, K. Park, Optimization of direct lysine decarboxylase biotransformation for cadaverine production with whole-Cell biocatalysts at high lysine concentration, J. Microbiol. Biotechnol. 25 (2015) 1108-1113.
[9] Y. M. Moon, S. Y. Yang, T. R. Choi, H. R. Jung, H. S. Song, Y Han, H. Y. Park, S. K. Bhatia, R. Gurav, K. Park, J. S. Kim, Y. H. Yang, Enhanced production of cadaverine by the addition of hexadecyltrimethylammonium bromide to whole cell system with regeneration of pyridoxal-5′-phosphate and ATP, Enzyme Microb. Technol. 127 (2019) 58-64.
[10] W. Ma, W. Cao, B. Zhang, K. Chen, Q. Liu, Y. Li, P. Ouyang, Engineering a pyridoxal 5′-phosphate supply for cadaverine production by using Escherichia coli whole-cell biocatalysis, Sci. Rep. 5 (2015) 15630.
[11] J. H. Kim, J. Kim, H. J. Kim, G. Sathiyanarayanan, S. K. Bhatia, H. S. Song, Y. K. Choi, Y. G. Kim, K. Parkd, Y. H. Yang, Biotransformation of pyridoxal 5′-phosphate from pyridoxal by pyridoxal kinase (pdxY) to support cadaverine production in Escherichia coli, Enzyme Microb. Technol. 104 (2017) 9-15.
[12] X. Zhang, I. S. Ng, J. S. Chang, Cloning and characterization of a robust recombinant azoreductase from Shewanella xiamenensis BC01, J. Taiwan Inst. Chem. Eng. 61 (2016) 97-105.
[13] Y. H. Kim, H. J. Kim, J. H. Shin, S. K. Bhatia, H. M. Seo, Y. G. Kim, Y. K. Lee, Y. H. Yang, Y. Park, Application of diethyl ethoxymethylenemalonate (DEEMM) derivatization for monitoring of lysine decarboxylase activity, J. Mol. 115 (2015) 151-154.
[14] V. Kumar, M. Sharma, B. R. Rakesh, C. K. Malik, S. Neelagiri, K. B. Neerupudi, P. Garg, S. Singh, Pyridoxal kinase: A vitamin B6 salvage pathway enzyme from Leishmania donovani, Int. J. Biol. Macromol. 119 (2018) 320-334.
[15] H. T. Kim, K. A. Baritugo, Y. H. Oh, S. M. Hyun, T. U. Khang, K. H. Kang, S. H. Jung, B. K. Song, K. Park, I. K. Kim, M. O. Lee, Y. Kam, Y. T. Hwang, S. J. Park, J. C. Joo, Metabolic engineering of Corynebacterium glutamicum for the high-level production of cadaverine that can be used for the synthesis of biopolyamide 510. ACS Sustainable Chem. Eng. 6 (2018) 5296-5305.

What is claimed is:

1. A recombinant microorganism for producing cadaverine, comprising a first nucleotide sequence encoding a first protein having lysine decarboxylase activity and a second nucleotide sequence encoding a second protein having pyridoxal kinase activity.

2. The recombinant microorganism according to claim 1, wherein the first nucleotide sequence comprises a sequence having at least 80% identity to SEQ ID NO. 1, and the second nucleotide sequence comprises a sequence having at least 80% identity to one of SEQ ID NOs. 2 to 4.

3. The recombinant microorganism according to claim 1, wherein the second nucleotide sequence further comprises a constitutive promoter for regulating expression of the second protein.

4. The recombinant microorganism according to claim 3, wherein the constitutive promoter is selected from the group consisting of J23100, J23101, J23102, J23103, J23104, J23105, J23106, J23107, J23108, J23109, J23110, J23111, J23112, J23113, J23114, J23115, J23116, J23117, J23118, J23119 and PlacI.

5. The recombinant microorganism according to claim 1, wherein the lysine decarboxylase is lysine decarboxylase 1 (CadA).

6. The recombinant microorganism according to claim 1, wherein the pyridoxal kinase is selected from the group consisting of PdxH, PdxK, PdxY, and any combination thereof.

7. The recombinant microorganism according to claim 1, which has at least one expression vector comprising at least one of the first nucleotide sequence and the second nucleotide sequence.

8. The recombinant microorganism according to claim 1, which has two expression vectors comprising the first nucleotide sequence and the second nucleotide sequence, respectively.

9. The recombinant microorganism according to claim 1, which is a recombinant microorganism of Escherichia, Klebsiella, Erwinia, Serratia, Providencia, Corynebacterium, or Brevibacterium.

10. The recombinant microorganism according to claim 1, which is a recombinant microorganism of E. coli BL21 or E. coli BL21(DE3).

11. A method of producing cadaverine, comprising:
culturing the recombinant microorganism of claim 1 in a medium for expression of the first protein and the second protein in the recombinant microorganism;
mixing the cultured recombinant microorganism with lysine in a solution to perform bioconversion of the lysine to the cadaverine; and
recovering the cadaverine from the solution.

12. The method according to claim 11, wherein the medium comprises an inducer for inducing the expression of the first protein in the recombinant microorganism.

13. The method according to claim 12, wherein the inducer is isopropyl β-d-1-thiogalactopyranoside (IPTG).

14. The method according to claim 13, wherein the inducer is in a concentration of from 5 μM to 50 μM.

15. The method according to claim 11, wherein the medium comprises a substrate of the pyridoxal kinase, and the substrate is selected from the group consisting of pyridoxamine (PM), pyridoxine (PN), pyridoxal (PL), pyridoxamine-5′-phosphate (PMP), pyridoxine-5′-phosphate (PNP), and any combination thereof.

16. The method according to claim 15, wherein the substrate is in a concentration of from 0.1 μM to 0.5 mM.

17. The method according to claim 11, wherein the lysine has a concentration of from 0.1M to 2.0 M.

18. The method according to claim 11, wherein the cultured recombinant microorganism is mixed in the solution after a concentration of the cultured recombinant microorganism represented by OD₆₀₀ in the medium achieves at least 0.5.

19. The method according to claim 11, further comprising separating the cultured recombinant microorganism from the medium, and adding the separated cultured recombinant microorganism into the solution to perform the mixing.

20. The method according to claim 11, wherein the bioconversion of the lysine to the cadaverine is performed in the cultured recombinant microorganism without adding external pyridoxal 5′-phosphate (PLP).
